(19) United States
(12) Patent Application Publication
(10) Pub. No.: US 2022/0314142 A1
(43) Pub. Date: Oct. 6, 2022

(54) IMPROVEMENTS TO WASH SOLUTIONS FOR PROTEIN A CHROMATOGRAPHY IN AN ANTIBODY PURIFICATION PROCESS

(71) Applicant: Pfizer Inc., New York, NY (US)

(72) Inventor: Russell Ian SHPRITZER, Tewksbury, MA (US)

(73) Assignee: Pfizer Inc., New York, NY (US)

(21) Appl. No.: 17/620,148

(22) PCT Filed: Jun. 9, 2020
(86) PCT No.: PCT/IB2020/056119

§ 371 Date: Dec. 17, 2021

Related U.S. Application Data
(60) Provisional application No. 62/869,093, filed on Jul. 1, 2019.

(51) Int. Cl. B01D 15/38; B01D 15/20; C07K 1/22; C07K 16/22

(52) U.S. Cl. CPC B01D 15/3809; B01D 15/203; C07K 1/22; C07K 16/22

(57) ABSTRACT

The invention relates to a Protein A chromatography step of a purification process for a therapeutic protein, wherein a load solution including the therapeutic protein is applied onto a Protein A chromatography medium. According to the invention, a solution comprising CaCl₂ is used as a wash solution for the Protein A chromatography medium for enhancing the removal of lipases, in particular phospholipase B-like 2 (PLBL2). The invention is of particular interest for the purification of CHO-expressed antibodies, such as tanezumab.

### TECHNICAL FIELD

The present invention relates to processes for the purification of therapeutic proteins, including a Protein A chromatography step.

### BACKGROUND OF THE INVENTION

Therapeutic proteins, in particular therapeutic antibodies, are produced in genetically engineered host cells, harvested from bioreactors and then purified under controlled multi-step processes designed to confer a high degree of purity to the final product.

One of the main challenges is the reduction of impurities, in particular residual host cell proteins (HCPs), that are proteins expressed by the host cells used for the production of the therapeutic protein.

While, from a regulatory standpoint, there may not be any defined acceptable level of HCP for all the biopharmaceutical products, it is required, on a case-by-case basis, to minimize the level of HCP in order to minimize any associated safety risk and negative effect on efficacy.

One of the conventional steps involved in the purification processes for therapeutic proteins consists of a Protein A chromatography step, wherein a load solution collected from the harvest step and comprising the therapeutic protein is applied to a Protein A chromatography medium, e.g. in the form of a resin arranged in a chromatography column.

Such Protein A chromatography column may be operated in a bind-and-elute mode. A conventional process of operating such Protein A chromatography column may therefore sequentially comprise
a step in which the therapeutic protein binds to the Protein A chromatography medium while the solutions including impurities flows through the Protein A chromatography medium;
a step in which in a subsequent operation, the Protein A chromatography medium is washed with one or more wash solution(s) such that more impurities are removed from the resin and separated from the therapeutic protein;
a step in which in a subsequent operation, the Protein A chromatography medium is contacted by an elution buffer for the recovery of the bound therapeutic protein.

In conventional processes, salt solutions such as sodium chloride (NaCl) solutions are used as wash solutions for removing HCPs. It is also known, e.g. from WO 2006/138553 and WO 2008/03120, to use divalent cation salt washes for the same purpose.

The invention is more specifically related to solutions to be used for washing the Protein A chromatography medium and enhancing the removal of certain HCPs.

The presence of specific HCPs, such as lipases and more specifically phospholipase B-like 2 (PLBL2), revealed at various stages of purification processes for Chinese Hamster Ovary (CHO)-expressed therapeutic antibodies, is of particular concern. PLBL2, for example, was found to interfere with molecular bonds of conventional excipients such as polysorbate, in particular polysorbate 80 (PS80) and polysorbate 20 (PS20), and thus affect the stability of the resulting drug product. Impact of PLBL2 on PS20 degradation is for example reported in the following publication: “Residual Host Cell Protein Promotes Polysorbate 20 Degradation in a Sulfatase Drug Product Leading to Free Fatty Acid Particles”—Nitin Dixit et al.—Journal of Pharmaceutical Sciences Volume 105, Issue 5, May 2016, Pages 1657-1666.

At certain levels, PLBL2 may potentially affect the immunogenicity of the product.

The immunogenic potential of PLBL2 and its potential impact on the immunogenicity of a monoclonal antibody were for example evaluated and reported in the following publication: “Specific Immune Response to Phospholipase B-Like 2 Protein, a Host Cell Impurity in Lebrikizumab Clinical Material”—Saloumeh Kadkhodayan Fischer et al. —The AAPS Journal (© 2016).

There is a need to address this problem of therapeutic antibody purification and it is an aim of the invention to provide wash solutions that are effective to drastically reduce the amount of lipases, in particular PLBL2, in the eluted pool recovered from the Protein A chromatography medium and in the drug product.

Specific challenges associated with removing PLBL2 in an antibody purification process are documented e.g. in the following article: “Investigating interactions between phospholipase B-Like 2 and antibodies during Protein A chromatography”—B. Tran et al.—Journal of Chromatography A 1438 (2016) 31-38.

### SUMMARY OF THE INVENTION

According to a first aspect of the present invention, a solution comprising CaCl₂ is used as a wash solution for the Protein A chromatography medium for enhancing the removal of lipases.

According to a further aspect of the present invention, a solution comprising CaCl₂ is used as a wash solution for the Protein A chromatography medium for enhancing the removal of PLBL2.

It has been found for example that such use of CaCl₂ wash solution, as compared to conventional wash solutions such as in particular NaCl solutions, can reduce the weight ratio of lipases, more specifically of PLBL2, to the therapeutic protein in the eluted pool and in the drug product by at least 2 fold, even at least 7 fold, or even at least 30 fold. In particular, such use of CaCl₂ wash solution can reduce the weight ratio of lipases, more specifically of PLBL2, while being at least as effective as conventional wash solutions, such as in particular NaCl solutions, for the overall reduction of HCPs.

In a further aspect of the invention, a process of purification of a therapeutic protein is provided, the process comprising a Protein A chromatography step wherein
a load solution comprising the therapeutic protein is contacted with a Protein A chromatography medium, whereby the therapeutic protein binds to the Protein A chromatography medium;
the Protein A chromatography medium is washed with at least one wash solution comprising CaCl₂ for enhancing the removal of lipases, in particular for enhancing the removal of PLBL2; and
the therapeutic protein is eluted from the Protein A chromatography medium.

In a further aspect of the invention, it is provided a pharmaceutical product including a therapeutic protein purified by a process according to the invention.

According to preferred embodiments of the invention, in all its aspects:
the concentration of CaCl₂ in the wash solution is between 0.25 M and 3 M, preferably between 0.25 M and 2 M, preferably between 0.25 M and 1 M, preferably between 0.25 M and 0.8 M, preferably between 0.4 M and 0.6 M, and still more preferably of about 0.5 M;
the wash solution comprising CaCl₂ further comprises Tris;
the concentration of Tris in the wash solution comprising CaCl₂ is between 40 mM and 60 mM, preferably of about 50 mM;
the pH of the wash solution comprising CaCl₂ is between 5 and 9, preferably between 6 and 8.5, preferably between 7 and 8, preferably about 7.5;
prior to washing the Protein A chromatography medium with the solution comprising CaCl₂, the Protein A chromatography medium is washed with a solution comprising Tris at a concentration between 40 mM and 60 mM, preferably of about 50 mM, and NaCl at a concentration between 120 mM and 180 mM, preferably of about 150 mM, and having a pH between 6 and 9, preferably of about 7.5;
after washing the Protein A chromatography medium with the solution comprising CaCl₂, the Protein A chromatography medium is washed with a solution comprising acetate at a concentration between 20 mM and 30 mM, preferably of about 25 mM, and having a pH between 4 and 7, preferably of about 5.5;
prior to loading and washing the Protein A chromatography medium, the Protein A chromatography medium is contacted by an equilibration buffer comprising Tris at a concentration between 40 mM and 60 mM, preferably of about 50 mM, and NaCl at a concentration between 120 mM and 180 mM, preferably of about 150 mM, and having a pH between 6 and 9, preferably of about 7.5;
after washing the Protein A chromatography medium, the Protein A chromatography medium is contacted by an elution buffer for eluting the therapeutic protein bound to the Protein A chromatography medium;
said elution buffer comprises acetate at a concentration between 20 mM and 30 mM, preferably of about 25 mM, and has a pH between 2.5 and 4.5, preferably of about 3.7;
the therapeutic protein is a therapeutic antibody;
the therapeutic antibody is recombinantly produced from CHO cells;
the therapeutic antibody is tanezumab.

According to preferred embodiments of the process according to the invention:
the therapeutic protein is subjected to further purification steps, including an anion exchange chromatography step and/or a cation exchange purification step;
the therapeutic protein is formulated for use as a medicament.

### DETAILED DESCRIPTION OF THE INVENTION

### Definitions

The following definitions will be used in the present description and claims:
the term “Protein A” encompasses Protein A recovered from a native source thereof, Protein A produced synthetically (e.g. by peptide synthesis, by recombinant techniques, etc.), and variants thereof which retain the ability to bind proteins which have a CH2/CH3 region;
the term “harvested solution” refers to a solution containing at least one target substance which is sought to be purified from other substances also present. The harvested solutions are often complex mixtures containing many biological molecules (such as proteins, antibodies, hormones, and viruses), small molecules (such as salts, sugars, lipids, etc.) and even particulate matter. While a typical harvested solution of biological origin may be an aqueous solution or suspension, it may also contain organic solvents used in earlier separation steps such as solvent precipitations, extractions, and the like. Examples of harvested solutions that may contain valuable biological substances amenable to the purification by various embodiments of the present invention include, but are not limited to, a culture supernatant from a bioreactor, a homogenized cell suspension, plasma, plasma fractions, and milk;
the term “load solution” means the solution derived from the harvested solution, which may have been further centrifuged, depth-filtered or otherwise pre-purified and which is loaded on the Protein A chromatography column;
the term “therapeutic protein” refers to the protein to be purified from the harvested solution;
the term “impurities” refers to materials in the harvested solution or load solution that are different from the therapeutic protein and are desirably excluded from the final therapeutic protein formulation. Typical impurities include nucleic acids, proteins (including HCPs, low molecular weight species and under disulfide bonded species), peptides, endotoxins, viruses and small molecules;
the term “lipase” refers to any enzyme that catalyzes the breakdown of fats;
the term “PLBL2” means the lipase phospholipase B-like 2 and fragments or variants that may be present in the harvested solution deriving from the lipase phospholipase B-like 2. PLBL2 according to the invention may be measured by Liquid Chromatography-tandem Spectrometry (LC-MS/MS) or with a PLBL2 ELISA (Enzyme-Linked Immuno Sorbent Assay) kit as known in the art. A PLBL2 ELISA is a sandwich-based ELISA that utilizes polyclonal antibodies that bind to and enable quantitation of PLBL2 in a solution such as Chinese hamster PLBL2;
the term “wash solution” refers to the liquid used to carry away impurities from the chromatography medium to which is bound the therapeutic protein. More than one wash solution can be employed sequentially, e.g., with the successive wash solutions having varying properties such as pH, conductivity, solvent concentration, etc., designed to dissociate and remove varying types of impurities that are non-specifically associated with the chromatography medium;
the term “elution buffer” refers herein to the liquid that is used to dissociate the therapeutic protein from the chromatography medium after it has been washed with one or more wash solution(s). The elution buffer acts to dissociate the therapeutic protein without denaturing it irreversibly. Typical elution buffers are well known in the chromatography art and may have higher concentrations of salts, free affinity ligands or analogs, or other substances that promote dissociation of the therapeutic protein from the chromatography medium. A typical mode of eluting may also be based on the pH of the elution buffer;
the term “eluted pool” refers to the liquid phase produced by the elution step and including the elution buffer and the proteins eluted from the chromatography medium;
the term “drug substance” refers to the therapeutic protein as an active pharmaceutical ingredient as obtainable by the processes of the present invention;
the term “drug product” or “pharmaceutical product” refers to a finished dosage form that contains the therapeutic protein in association with excipients;
the term “excipients” means the constituents of the final therapeutic protein formulation, which are not the therapeutic protein. The excipients typically include protein stabilizers, surfactants, amino-acids e.g. contributing to protein stabilization, etc. . . . ;
unless stated otherwise, the term “about” associated with a numeral value means within a range of ±5% of said value.

The present invention relates to solutions comprising CaCl₂. Such solutions may be used as wash solutions for a Protein A chromatography medium used in a method to purify therapeutic proteins, in particular therapeutic antibodies.

Examples of therapeutic antibodies to be purified in a method according to the invention include therapeutic antibodies recombinantly produced from CHO cells. Preferably, tanezumab is the therapeutic antibody.

Preferably the use of solutions comprising CaCl₂ in the methods of the invention enhance the removal of PLBL2. The presence of PLBL2 may be measured by methods known in the art. For example, the presence of PLBL2 may be measured by Liquid Chromatography-tandem Spectrometry (LC-MS/MS) or with a PLBL2 ELISA (Enzyme-Linked Immuno Sorbent Assay) kit.

Accordingly, in one embodiment of the present invention PLBL2 is measured by Liquid Chromatography-tandem Spectrometry (LC-MS/MS).

In another embodiment of the present invention PLBL2 is measured with a PLBL2 ELISA (Enzyme-Linked Immuno Sorbent Assay).

Such enhancement of removal may be such that the amount of PLBL2 present in the eluted pool is at least 2 fold, 7 fold, or in some preferred embodiments 30 fold, less than in a standard process. A standard process is hereinafter defined in the “Example” section, in relation to the testing results shown in Tables 2 and 3.

Preferably, the amount of therapeutic antibody is not substantially diminished during the removal of PLBL2 by uses and processes according to the invention. For example, according to the invention the amount of therapeutic antibody present in the eluted pool may be about the same as compared to a standard process. In another example, the amount of therapeutic antibody present in the eluted pool may be higher as compared to a standard process.

Accordingly, suitable solutions comprising CaCl₂ comprise, but are not limited, to the following exemplary solution: 50 mM Tris, 0.5 M CaCl₂, pH 7.5.

Purification processes for therapeutic proteins using a Protein A chromatography step are well-known in the art and can be employed with the solutions comprising CaCl₂ of the invention.

According to the invention, a Protein A chromatography column may be operated in a bind-and-elute mode. A process of operating such Protein A chromatography column in a bind-and-elute may therefore comprise sequentially
a step in which the therapeutic protein binds to the Protein A chromatography medium while the solutions including impurities flows through the Protein A chromatography medium;
a step in which in a subsequent operation, the Protein A chromatography medium is washed with one or more wash solution(s) such that more impurities are removed from the resin and separated from the therapeutic protein;
a step in which in a subsequent operation, the Protein A chromatography medium is contacted by an elution buffer for the recovery of the bound therapeutic protein.

It has surprisingly been found that such use of CaCl₂ wash solution with a Protein A chromatography column operated in a bind-and-elute mode can reduce the weight ratio of PLBL2 to the therapeutic protein in the eluted pool to preferably less than 10 ng/mg. More preferably, it can reduce the weight ratio of PLBL2 to the therapeutic protein in the eluted pool to less than 8 ng/mg, and still more preferably to less than 6 ng/mg.

In particular, such use of CaCl₂ wash solution can reduce the weight ratio of PLBL2 to the therapeutic protein in the eluted pool to less than 10 ng/mg while being at least as effective as other conventional wash solutions, such as in particular NaCl solutions, for the general reduction of HCPs.

Other conventional wash solutions can optionally be used in addition to the CaCl₂ wash solution. For example, a wash solution including 50 mM Tris and 150 mM NaCl at pH 7.5 may preferably be used prior to the CaCl₂ wash solution. Still more preferably, a further wash solution including 25 mM Acetate at pH 5.5 may be used after the CaCl₂ wash solution.

By “general reduction of HCPs”, it is meant that the weight ratio of HCP species present in the eluted pool to the therapeutic protein is reduced. The general reduction in HCPs can be measured by methods known in the art, such as HCP ELISA (usually used as the primary tool) and LC-MS/MS.

Thus, in one aspect of the present invention, a process of purification of a therapeutic protein is provided, comprising a Protein A chromatography step wherein
the load solution comprising the therapeutic protein is contacted with a Protein A chromatography medium, whereby the therapeutic protein binds to the Protein A chromatography medium;
the Protein A chromatography medium is washed with at least one wash solution comprising CaCl₂ for enhancing the removal of phospholipase B-like 2 (PLBL2); and
the therapeutic protein is eluted from the Protein A chromatography medium.

Preferably, the concentration of CaCl₂ in said at least one wash solution is between about 0.4 M and about 0.5 M, preferably about 0.5 M.

Preferably, the wash solution comprising CaCl₂ further comprises Tris, preferably at a concentration of about 50 mM.

Preferably, the wash solution comprising CaCl₂ has a pH of about 7.5.

In a further aspect of the present invention, the use of a solution comprising CaCl₂ as a wash solution for the Protein A chromatography medium for enhancing the removal of phospholipase B-like 2 (PLBL2) is provided.

In a preferred embodiment of the invention, prior to washing the Protein A chromatography medium with the solution comprising CaCl₂, the Protein A chromatography medium is washed with a solution comprising Tris at a concentration of about 50 mM and NaCl at a concentration of about 150 mM, and having a pH of about 7.5.

In a preferred embodiment of the invention, after washing the Protein A chromatography medium with the solution comprising CaCl₂, the Protein A chromatography medium is washed with a solution comprising acetate at a concentration of about 25 mM and having a pH of about 5.5.

In a preferred embodiment of the invention, prior to loading and washing the Protein A chromatography medium, the Protein A chromatography medium is contacted by an equilibration buffer comprising Tris at a concentration of about 50 mM and NaCl at a concentration of about 150 mM, and having a pH of about 7.5.

In a preferred embodiment of the invention, after washing the Protein A chromatography medium, the Protein A chromatography medium is contacted by an elution buffer for eluting the therapeutic protein bound to the Protein A chromatography medium, said elution buffer comprising acetate at a concentration of about 25 mM and having a pH of about 3.7.

In a preferred embodiment of the invention, after washing the Protein A chromatography medium, the Protein A chromatography medium is contacted by an elution buffer for eluting the therapeutic protein bound to the Protein A chromatography medium and an eluted pool comprising the therapeutic protein is collected.

In a preferred embodiment, the therapeutic protein is a therapeutic antibody, preferably a therapeutic antibody expressed in CHO cells (or recombinantly produced from CHO cells), and more preferably tanezumab.

Tanezumab is an anti-nerve growth factor (NGF) antibody. The sequences for tanezumab are provided in Table 1 below. Tanezumab is described, as antibody E3, in WO2004/058184.

TABLE 1

| SEQ ID NO: | Sequence |
|---|---|
| 1 | Variable heavy chain region: |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVS |
| 2 | Variable light chain region: |
| | DIQMTQSPSSLSASVGDRVTITCRASQSISNNLNWYQQKPGKAPKLL |
| | IYYTSRFHSGVPSRFSGSGSGTDFTFTISSLQPEDIATYYCQQEHTLP |
| | YTFGQGTKLEIKRT |
| 3 | Extended HCDR1: |
| | GFSLIGYDLN |
| 4 | Extended HCDR2: |
| | IIWGDGTTDYNSAVKS |
| 5 | Extended HCDR3: |
| | GGYWYATSYYFDY |
| 6 | Extended LCDR1: |
| | RASQSISNNLN |
| 7 | Extended LCDR2: |
| | YTSRFHS |
| 8 | Extended LCDR3: |
| | QQEHTLPYT |
| 9 | Heavy chain*: |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSNFGTQTYTCNVDHKPSNTKVDKTVERKCCVECPPCPAP |
| | PVAGPSVFLFPPKPKDTLMISRTPEVTCVVVDVSHEDPEVQFNWYV |
| | DGVEVHNAKTKPREEQFNSTFRVVSVLTVVHQDWLNGKEYKCKVS |
| | NKGLPSSIEKTISKTKGQPREPQVYTLPPSREEMTKNQVSLTCLVKG |
| | FYPSDIAVEWESNGQPENNYKTTPPMLDSDGSFFLYSKLTVDKSRW |
| | QQGNVFSCSVMHEALHNHYTQKSLSLSPGK |
| 10 | Light chain: |
| | DIQMTQSPSSLSASVGDRVTITCRASQSISNNLNWYQQKPGKAPKLL |
| | IYYTSRFHSGVPSRFSGSGSGTDFTFTISSLQPEDIATYYCQQEHTLP |
| | YTFGQGTKLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC |
| 11 | Heavy chain (C-terminal lysine (K) processed) |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSNFGTQTYTCNVDHKPSNTKVDKTVERKCCVECPPCPAP |
| | PVAGPSVFLFPPKPKDTLMISRTPEVTCVVVDVSHEDPEVQFNWYV |
| | DGVEVHNAKTKPREEQFNSTFRVVSVLTVVHQDWLNGKEYKCKVS |
| | NKGLPSSIEKTISKTKGQPREPQVYTLPPSREEMTKNQVSLTCLVKG |
| | FYPSDIAVEWESNGQPENNYKTTPPMLDSDGSFFLYSKLTVDKSRW |
| | QQGNVFSCSVMHEALHNHYTQKSLSLSPG |

[* C-terminal lysine (K) of the heavy chain amino acid sequence of SEQ ID NO: 9 is optional]

A further aspect of the present invention provides a pharmaceutical product including a therapeutic protein purified by a process of the invention described herein. In a preferred embodiment, the therapeutic protein is a therapeutic antibody, preferably a therapeutic antibody expressed in CHO cells (or recombinantly produced from CHO cells), and more preferably tanezumab.

In a preferred embodiment the level of PLBL2 in the product is less than or equal to about 10 ng/mg as measured by LC-MS/MS or PLBL2 ELISA. In some embodiments, the level of PLBL2 is less than about 9, 8, 7, 6, 5, 4, 3, 2 or 1 ng/mg. In a preferred embodiment the level of PLBL2 is less than about 2 or 1 ng/mg.

A further aspect of the present invention provides a composition obtainable by a process of the invention described herein. In a preferred embodiment, the therapeutic protein is a therapeutic antibody, preferably a therapeutic antibody expressed in CHO cells (or recombinantly produced from CHO cells), and more preferably tanezumab.

In a preferred embodiment the level of PLBL2 in the composition is less than or equal to about 10 ng/mg as measured by LC-MS/MS or PLBL2 ELISA. In some embodiments, the level of PLBL2 is less than about 9, 8, 7, 6, 5, 4, 3, 2 or 1 ng/mg. In a preferred embodiment the level of PLBL2 is less than about 2 or 1 ng/mg.

A further aspect of the present invention provides a pharmaceutical composition comprising tanezumab, wherein the level of PLBL2 in the composition is less than or equal to about 10 ng/mg as measured by LC-MS/MS or PLBL2 ELISA.

In some embodiments, the level of PLBL2 is less than about 9, 8, 7, 6, 5, 4, 3, 2 or 1 ng/mg. In a preferred embodiment the level of PLBL2 is less than about 2 or 1 ng/mg.

The compositions of the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

Indeed, as used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some embodiments, these agents are formulated for administration by injection (e.g., intraperitoneally, intravenously, subcutaneously, intramuscularly, intraarticularly etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments the compositions comprising tanezumab, is a composition as described in WO2010/032220, herein incorporated by reference.

In some embodiments, the composition is a liquid formulation and comprises tanezumab at a concentration of about 2.5 mg/ml, 5 mg/ml, 10 mg/ml or 20 mg/ml; and a histidine buffer.

In some embodiments, the formulation further comprises a surfactant which may be polysorbate 20. In some embodiments, the formulation further comprises trehalose dehydrate or sucrose. In some embodiments, the formulation further comprises a chelating agent, which may be EDTA; in some embodiments, disodium EDTA. In some embodiments, the formulation is of pH 6.0±0.3.

In some embodiments, the formulation comprises about 2.5 mg/ml, 5 mg/ml, 10 mg/ml or 20 mg/ml tanezumab; about 10 mM histidine buffer; about 84 mg/ml trehalose dehydrate; about 0.1 mg/ml Polysorbate 20; about 0.05 mg/ml disodium EDTA; wherein the formulation is of a pH 6.0±0.3.

In some embodiments the formulation comprises about 2.5 mg/ml or 5 mg/ml. In some embodiments, the formulation has a total volume of about 1 ml.

In some embodiments the formulation is contained in a glass or plastic vial or syringe. In some embodiments the formulation is contained in a pre-filled glass or plastic vial or syringe. In some embodiments the formulation is contained in a self-injector syringe.

In some embodiments the compositions are administered via to a subject via systemic administration (e.g., intravenous or subcutaneous administration). Preferably the compositions are administered via subcutaneous injection.

In some embodiments the compositions or pharmaceutical product of the invention comprising tanezumab are for use in methods of treatment of pain, including osteoarthritis pain, low back pain, and cancer pain. The use of tanezumab in the treatment of osteoarthritis pain and low back pain is further described in International Patent Application Nos. PCT/IB2020/050611 and PCT/IB2020/051281, respectively, the contents of which are herein incorporated by reference. In embodiments that refer to methods of treatment as described herein, such embodiments are also further embodiments for use in that treatment, or alternatively for the manufacture of a medicament for use in that treatment.

### Example

The invention will now be further illustrated by the following Example, corresponding to a purification process applied to a specific therapeutic monoclonal antibody and evaluated with various Protein A chromatography wash strategies. The Example is provided for illustrative purpose only and should not be construed as limiting the scope of the invention.

In the illustrative Example, the therapeutic protein is tanezumab (sequence shown in Table 1 above and further described in WO2004/058184), an antibody that specifically binds to NGF (Nerve Growth Factor) and that can be used as a treatment for pain, including osteoarthritis pain, low back pain, and cancer pain.

In this Example, the therapeutic protein, present in a load solution collected from a bioreactor, is purified by a multi-step purification process that sequentially includes
an initial centrifugation step;
an initial depth filtration step;
a Protein A affinity chromatography step, that is run in a chromatography column comprising a Protein A chromatography medium such as a MabSelect™, MabSelect SuRe™ or MabSelect SuRe LX™ resin, available from GE Healthcare, or an Eshmuno™ A or ProSep™ Ultra Plus resin, available from Millipore Sigma. The resin is initially equilibrated with an equilibration buffer. The column is then loaded such that the target antibody binds to the resin. The resin is subsequently washed with one or more wash solution(s) and an elution buffer is then applied, whereby the target antibody is eluted from the resin in an elution pool. The primary objectives of the Protein A chromatography step include the product capture from clarified, cell-free conditioned medium and the separation of tanezumab from process-derived impurities;
a low pH virus inactivation step;
an anion exchange (AEX) chromatography step, that is run in a chromatography column comprising a medium such as a Q Sepharose™, Capto™ Q or Capto Q ImpRes™ resin, available from GE Healthcare, a TOYOPEARL GigaCap™ Q-650M resin from Tosoh Bioscience, or a Fractogel™ EMD TMAE HiCap (M) or Eshmuno™ Q resin from Millipore Sigma;
a cation exchange (CEX) chromatography step, that is run in a chromatography column comprising a medium such as a SP Sepharose™ FF, Capto S or Capto SP ImpRes resin, available from GE Healthcare, a TOYOPEARL GigaCap™ S-650M resin from Tosoh Bioscience, or a Fractogel™ EMD S03-(M) or Eshmuno™ S resin from Millipore Sigma;
a virus filtration step using a Planova™ filter, available from Asahi Kasei, or a ViresolvePro™ filter from Millipore Sigma;
an ultrafiltration/diafiltration step; and
a final formulation and filtration step.

Protein a Chromatography Wash Buffer Evaluation

Three batches (Pilot Batch 1-3) were run in a 500 L pilot plant, using the above purification process, with two sequential washes for the Protein A chromatography column. The first wash solution is a 50 mM phosphate, 1M NaCl, pH 7 solution and the second wash solution is a 25 mM acetate, pH 5.5 solution. The elution is performed with a 25 mM acetate, pH 3.7 elution buffer. This process is hereinafter referred to as the “standard process”.

Characterization work was performed on these three batches, including analysis of HCP by Liquid Chromatography-tandem Spectrometry (LC-MS/MS) conventional testing techniques. The LC-MS/MS results revealed the presence of a single HCP, phospholipase B-like 2 (PLBL2), remaining in the tanezumab drug substance.

All three batches were run with the same process, except that Batch 3 included a depth filter after the low pH virus inactivation and neutralization of the Protein A pool. The depth filter was added to remove particulates and prevent plugging of the subsequent 0.2 μm filter.

The results of this testing are shown in Table 2.

TABLE 2
Levels of PLBL2 Detected in Pilot Batches by LC-MS/MS (ng/mg)

| Process Step | Pilot Batch 1 | Pilot Batch 2 | Pilot Batch 3 |
|---|---|---|---|
| Protein A Load | 596 | 667 | 426 |
| Protein A | 292 | 379 | 90 |
| Elution Pool | | | |
| AEX Load | 301 | 314 | 35-47 |
| AEX Pool | 118 | 180 | 32 |
| CEX Pool | 126 | 84 | <10 |
| Drug Substance | 42 | 46 | <10 |

Results from additional testing of the pools using a commercially available PLBL2 ELISA (Enzyme-Linked Immuno Sorbent Assay) kit are shown in Table 3.

TABLE 3
Levels of PLBL2 Detected in Pilot
Batches by PLBL2 ELISA (ng/mg)

| Process Step | Pilot Batch 1 | Pilot Batch 2 | Pilot Batch 3 |
|---|---|---|---|
| Protein A Load | 272 | 490 | 293 |
| Protein A | 70 | 100 | 31 |
| Elution Pool | | | |
| AEX Load | 80 | 146 | 13 |
| AEX Pool | 43 | 42 | 11 |
| CEX Pool | 33 | 32 | 6 |
| Drug Substance | 28 | 39 | 7 |

The PLBL2 ELISA results are very consistent with the LC-MS/MS, showing that a low level of PLBL2 is not cleared by the process. The inclusion of the depth filter in Batch 3 did result in lower levels of PLBL2 in the drug substance, but it was determined that a more robust solution to remove PLBL2 should be identified.

Based on these results, a new wash buffer containing 0.5 M CaCl₂ was implemented which successfully removed PLBL2. The details of the development of this wash step are described in the following.

It has been observed that a wash buffer containing 0.5 M CaCl₂ generally provides more robust impurity removal than a wash buffer containing 1 M NaCl.

An initial wash screen, using Pilot Batch 1 Protein A load, was performed with 0.6 mL mini-columns to evaluate whether incorporation of a 0.5 M CaCl₂ wash buffer could effectively reduce the levels of PLBL2.

Table 4 below shows the different wash conditions that were evaluated and the HCP levels measured in the elution pools.

Condition 1 represents the standard process wash scheme used for the three Pilot batches.

Condition 2 adds a 0.5 M CaCl₂ wash and replaces the phosphate in Wash 1 with Tris to prevent precipitation due to calcium and phosphate coming into contact.

Condition 3 is the same as Condition 2, except the 1 M NaCl in the equilibration and Wash 1 buffers is reduced to 150 mM NaCl.

TABLE 4
Initial Protein A Chromatography Step Wash Screen

| Step | Load | Condition 1 | Condition 2 | Condition 3 |
|---|---|---|---|---|
| Equilibration/Wash 1 | | 50 mM Phosphate, | 50 mM Tris, | 50 mM Tris, |
| | | 1M NaCl, | 1M NaCl, | 150 mM NaCl, |
| | | pH 7.0 | pH 7.5 | pH 7.5 |
| Wash 2 | | N/A | 50 mM Tris, | 50 mM Tris, |
| | | | 0.5M CaCl₂, | 0.5M CaCl₂, |
| | | | pH 7.5 | pH 7.5 |
| Wash 3 | | 25 mM Acetate, | 25 mM Acetate, | 25 mM Acetate, |
| | | pH 5.5 | pH 5.5 | pH 5.5 |
| HCP ELISA (ng/mg) | 131,065 | 657 | 310 | 324 |
| PLBL2 by | 435 | 100 | <10 | <10 |
| LC-MS/MS (ng/mg) | | | | |
| PLBL2 by | 268 | 39 | 1 | 1 |
| PLBL2 ELISA | | | | |
| (ng/mg) | | | | |

Both Condition 2 and Condition 3, with the inclusion of a CaCl₂ wash, result in a significant reduction in PLBL2 as compared to the 1 M NaCl wash. Additionally, the removal of total HCP as measured by ELISA is moderately improved with the CaCl₂ wash. No notable difference was seen in this regard between Condition 2 and Condition 3, with the reduction of the NaCl concentration from 1 M to 150 mM.

Based on the results of the initial wash screen, the CaCl₂ wash was evaluated head-to-head with the NaCl wash on qualified lab-scale columns using load materials from all three pilot-scale batches.

The Protein A pools from each run were subsequently processed over the AEX column.

The PLBL2 ELISA results are shown in Table 5, with the results for Pilot Batch 1 also confirmed by LC-MS/MS.

TABLE 5
NaCl Wash vs. CaCl₂ Wash: levels of PLBL2
detected by LC-MS/MS and PLBL2 ELISA (ng/mg)

| | | Pilot Batch | | | |
|---|---|---|---|---|---|
| | | 1 Load | | Pilot Batch | Pilot Batch |
| | | LC- | | 2 Load | 3 Load |
| | Pool | MS/MS | ELISA | ELISA | ELISA |
| Load | Protein A Load | 380 | 332 | 518 | 314 |
| 1 M NaCl | Protein A Pool | 86 | 63 | 110 | 67 |
| Wash | AEX Pool | 75 | 50 | 53 | 48 |
| 0.5 M | Protein A Pool | <10 | 2 | 3 | 2 |
| CaCl₂ | AEX Pool | <10 | 1 | 1 | 1 |
| Wash | | | | | |

The results demonstrate that the CaCl₂ wash consistently reduces the level of PLBL2 to less than 10 ng/mg using the load material from all three pilot batches.

To further confirm the robustness of the CaCl₂ wash, multiple runs were performed with varying concentrations of CaCl₂. The results are shown in Table 6 and confirm the robustness of PLBL2 removal even at lower levels of CaCl₂.

TABLE 6
Impact of CaCl₂ Concentration on the levels of PLBL2
Detected by PLBL2 ELISA (ng/mg) in the Protein A pool

| | PLBL2 ELISA |
|---|---|
| Wash Condition | (ng/mg) |
| Load | 328 |
| 0.25M CaCl₂ | 10 |
| 0.4M CaCl₂ | 4 |
| 0.5M CaCl₂ | 9 |

Table 7 summarizes a final process (2^{nd} column of the table) found to be optimal for the tanezumab Protein A chromatography step, and highlights the optimizations that were made from the standard process (1^{st} column).

TABLE 7
Summary of Optimizations to the Standard Process
for the Protein A Affinity Chromatography Step

| Step | Standard process | Optimized process |
|---|---|---|
| Equilibration | 50 mM Phosphate, 1M NaCl, pH 7.0 | 50 mM Tris, 150 mM NaCl, pH 7.5 |
| Load | Clarified, Cell-free Conditioned Medium | |
| Wash 1 | 50 mM Phosphate, 1M NaCl, pH 7.0 | 50 mM Tris, 150 mM NaCl, pH 7.5 |
| Wash 2 | 25 mM Acetate, pH 5.5 | 50 mM Tris, 0.5M Calcium Chloride, pH 7.5 |
| Wash 3 | N/A | 25 mM Acetate, pH 5.5 |

| | |
|---|---|
| Elution | 25 mM Acetate, pH 3.7 |

As further confirmation of the ability of the CaCl₂ wash to clear PLBL2, an additional pilot-scale batch was run using the optimized process as outlined in Table 7. The results are shown in Table 8 and demonstrate that PLBL2 is brought to below the quantitation limit of the PLBL2 ELISA assay.

TABLE 8
Total Level of HCP Detected in Final
Pilot Batch by PLBL2 ELISA (ng/mg)

| Process Step | PLBL2 by ELISA |
|---|---|
| Protein A Load | 526 |
| Protein A Elution Pool | 2 |
| AEX Load | NMT 1 |
| AEX Pool | NMT 1 |
| CEX Pool | NMT 1 |
| Drug Substance | NMT 1 |

NMT = Not More Than

In conclusion, the above-described experiments demonstrate the consistent reduction of PLBL2 levels associated with the use of CaCl₂ in a wash solution for the Protein A affinity chromatography step.

Although the disclosed teachings have been described with reference to various applications, processes and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. Use, in a Protein A chromatography step of a purification process for a therapeutic protein, wherein a load solution including the therapeutic protein is applied onto a Protein A chromatography medium, of a solution comprising CaCl₂ as a wash solution for the Protein A chromatography medium for enhancing the removal of lipases.

2. Use according to claim 1 for enhancing the removal of phospholipase B-like 2 (PLBL2).

3. Use according to claim 1 or 2, wherein the concentration of CaCl₂ in the wash solution is between 0.25 M and 3 M, preferably between 0.25 M and 2 M, preferably between 0.25 M and 1 M, preferably between 0.25 M and 0.8 M, preferably between 0.4 M and 0.6 M, and still more preferably of about 0.5 M.

4. Use according to claim 1, wherein the wash solution comprising CaCl₂ further comprises Tris.

5. Use according to claim 4, wherein the concentration of Tris in the wash solution comprising CaCl₂ is between 40 mM and 60 mM, preferably of about 50 mM.

6. Use according to claim 1, wherein the pH of the wash solution comprising CaCl₂ is between 5 and 9, preferably between 6 and 8.5, preferably between 7 and 8, preferably about 7.5.

7. Use according to claim 1, wherein, prior to washing the Protein A chromatography medium with the solution comprising CaCl₂, the Protein A chromatography medium is washed with a solution comprising Tris at a concentration between 40 mM and 60 mM, preferably of about 50 mM, and NaCl at a concentration between 120 mM and 180 mM, preferably of about 150 mM, and having a pH between 6 and 9, preferably of about 7.5.

8. Use according to claim 1, wherein, after washing the Protein A chromatography medium with the solution comprising CaCl₂, the Protein A chromatography medium is washed with a solution comprising acetate at a concentration between 20 mM and 30 mM, preferably of about 25 mM, and having a pH between 4 and 7, preferably of about 5.5.

9. Use according to claim 1, wherein, prior to loading and washing the Protein A chromatography medium, the Protein A chromatography medium is contacted by an equilibration buffer comprising Tris at a concentration between 40 mM and 60 mM, preferably of about 50 mM, and NaCl at a concentration between 120 mM and 180 mM, preferably of about 150 mM, and having a pH between 6 and 9, preferably of about 7.5.

10. Use according to claim 1, wherein, after washing the Protein A chromatography medium, the Protein A chromatography medium is contacted by an elution buffer for eluting the therapeutic protein bound to the Protein A chromatography medium.

11. Use according to claim 10, wherein said elution buffer comprises acetate at a concentration between 20 mM and 30 mM, preferably of about 25 mM, and has a pH between 2.5 and 4.5, preferably of about 3.7.

12. Use according to claim 1, wherein the therapeutic protein is a therapeutic antibody.

13. Use according to claim 12, wherein the therapeutic antibody is recombinantly produced from CHO cells.

14. Use according to claim 13, wherein the therapeutic antibody is tanezumab.

15. Process of purification of a therapeutic protein, comprising a Protein A chromatography step wherein
a load solution comprising the therapeutic protein is contacted with a Protein A chromatography medium, whereby the therapeutic protein binds to the Protein A chromatography medium;
the Protein A chromatography medium is washed with at least one wash solution comprising CaCl₂ for enhancing the removal of lipases; and
the therapeutic protein is eluted from the Protein A chromatography medium.

16. Process according to claim 15, wherein the Protein A chromatography medium is washed with at least one wash solution comprising CaCl₂ for enhancing the removal of phospholipase B-like 2 (PLBL2).

17. Process according to claim 15, wherein the concentration of CaCl₂ in the wash solution is between 0.25 M and 3 M, preferably between 0.25 M and 2 M, preferably between 0.25 M and 1 M, preferably between 0.25 M and 0.8 M, preferably between 0.4 M and 0.6 M, and still more preferably of about 0.5 M.

18-29. (canceled)

30. Pharmaceutical product including a therapeutic protein purified by a process according to claim 15.

31. Pharmaceutical product of claim 30, wherein the level of PLBL2 in the product is less than or equal to about 10 ng/mg as measured by LC-MS/MS or PLBL2 ELISA.

32-34. (canceled)

35. Pharmaceutical composition comprising tanezumab, wherein the level of PLBL2 in the composition is less than or equal to about 10 ng/mg as measured by LC-MS/MS or PLBL2 ELISA.

36-37. (canceled)


SEQUENCE LISTING (11 sequences)

SEQ ID NO: 1 - protein, 120 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVS

SEQ ID NO: 2 - protein, 109 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRT

SEQ ID NO: 3 - protein, 10 aa, Artificial Sequence
  Synthetic Construct
GFSLIGYDLN

SEQ ID NO: 4 - protein, 16 aa, Artificial Sequence
  Synthetic Constuct
IIWGDGTTDYNSAVKS

SEQ ID NO: 5 - protein, 13 aa, Artificial Sequence
  Synthetic Construct
GGYWYATSYYFDY

SEQ ID NO: 6 - protein, 11 aa, Artificial Sequence
  Synthetic Construct
RASQSISNNLN

SEQ ID NO: 7 - protein, 7 aa, Artificial Sequence
  Synthetic Construct
YTSRFHS

SEQ ID NO: 8 - protein, 9 aa, Artificial Sequence
  Synthetic Construct
QQEHTLPYT

SEQ ID NO: 9 - protein, 447 aa, Artificial Sequence
  Synthetic Construct
  MISC_FEATURE (447)..(447): C-terminal lysine (K) at position 447 is optional
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG